(19) United States
(12) Patent Application Publication
(10) Pub. No.: US 2016/0369308 A1
(43) Pub. Date: Dec. 22, 2016

(54) METHOD FOR THE PRODUCTION OF LIPIDS BY MICROORGANISMS, AND USE OF SAID LIPIDS

(71) Applicants: AVRIL, Paris (FR); UNIVERSITE PARIS-SUID, Orsay (FR)

(72) Inventors: FRANCE THEVENIEAU, CHARTRES (FR); MARIE-JOËLLE VIROLLE, BURES SUR YVETTE (FR); THIERRY DULERMO, SAINT GERMAIN EN LAYE (FR); MAXIME VERGNE, LINXE (FR)

(73) Assignees: AVRIL, PARIS (FR); UNIVERSITE PARIS-SUD, ORSAY (FR)

(21) Appl. No.: 14/898,933

(22) PCT Filed: Jun. 20, 2014
(86) PCT No.: PCT/EP2014/063075

§ 371 Date: Dec. 16, 2015

(30) Foreign Application Priority Data
Jun. 21, 2013 (EP) 13305857.8

(51) Int. Cl. C12P 7/64; C12N 1/32; C10L 1/02; C07C 69/533; C07C 69/30; C12N 1/20; C12N 1/38

(52) U.S. Cl. CPC C12P 7/6463; C12N 1/20; C12N 1/32; C12N 1/38; C07C 69/533; C07C 69/30; C10L 1/02; C10L 2200/0476; C10L 2290/26

(57) ABSTRACT

The invention relates to the use of glycerol, or a C3-C5 sugar, as the main carbon source during the culturing of actinomyces bacteria for the production of lipids by said bacteria.

### CROSS REFERENCE TO RELATED APPLICATIONS

This application is the National Phase filing of International Appln. No. PCT/EP2014/063075, filed 20 Jun. 2014, and claims priority benefit of EP Appln. No. 13305857.8, filed 21 Jun. 2013, the entireties of which applications are incorporated herein by reference for all purposes.

### FIELD OF THE INVENTION

The present invention relates to the technical field of the production of lipids by microorganisms, and in particular to the use of said lipids.

### BACKGROUND OF THE INVENTION

The use of renewable biological materials for the production of biofuels is generally motivated by reducing the impacts on the climate and on food production, making the supply of fuels secure, and other economic factors.

Biological resources which cannot be used for human food or for livestock feed and which can be produced in an environmentally friendly manner are of increasing interest. Biodiesel can be produced from oleaginous crops, from animal fats or from recycled fats.

Since it is known that algae and certain microorganisms are capable of producing and/or accumulating lipids, the use thereof as a source of oil for biodiesel has also been suggested.

Campbell 2008 and Strobel et al. 2008 have described the optimization of the conditions for culturing algae and fungi in the various types of bioreactors in order to maximize lipid and fatty acid yields for the refining of biofuels.

An alternative to the photosynthetic production of lipids consists in using heterotrophic organisms which produce lipids from organic molecules (such as sugars) without light.

Oils derived from single-celled organisms have conventionally been used as special products, for example in health foods, and not as basic chemical products.

This type of production unfortunately has relatively low yields and the final product becomes expensive.

A new generation of methods using less expensive raw materials for the production of lipids by heterotrophic microorganisms has been proposed in certain recent patent publications.

Application WO 2009/034217 has described a method of fermentation for producing paraffins, fatty acids and alcohols by means of waste and microorganisms.

Application WO 2009/046375 describes the conversion of polysaccharides derived from biomass into monosaccharides and oligosaccharides, and their conversion into biofuels using recombinant microorganisms comprising exogenous genes which allow said microorganism to grow on the polysaccharide as sole carbon source.

Application US 2009/0064567 describes the production of biological oils by heterotrophic fermentation of microorganisms using raw materials containing cellulose as main carbon source.

Application WO 2009/011480 A1 describes the production of biological oils from depolymerized cellulose-based matter by microalgae and fungi.

Moreover, document WO 2009/009391 A2 describes the preparation of fatty esters by first producing an alcohol composition and supplying this product to a host for the production of a fatty ester.

Application US2011294173 describes a method for the production of lipids from bacteria of the Streptomyces genus by providing organic waste as carbon-based source.

These documents do not however solve a major problem: the lipid yield produced by the various microorganisms.

Thus, the reduction in production costs and the increase in the amount of lipid remain as yet unsolved problems.

The objective of the invention is to overcome these drawbacks.

### SUMMARY OF THE INVENTION

One of the objectives of the invention is to provide a method for the production of lipids which is inexpensive and which has a high yield.

Another objective of the invention is to provide culture media which make it possible to carry out the method using microorganisms.

The description relates to the use of glycerol, or a C₃-C₅ sugar, as the main carbon source during the culturing of actinomycete bacteria for the production of lipids by said bacteria, said bacteria being cultured in a first medium enriched with phosphate and/or with nitrogen, and then cultured in a second culture medium which is phosphate- and/or nitrogen-poor.

Thus, the invention relates to the use of glycerol, or a C₃-C₅ sugar, as main carbon source during the culturing of actinomycete bacteria for the production of lipids by said bacteria, said bacteria being cultured in a first medium enriched with phosphate, then cultured in a second culture medium which is phosphate-poor.

### DETAILED DESCRIPTION OF THE INVENTION

The invention is based on the surprising finding made by the inventors that actinomycete bacteria, cultured in the presence of glycerol as carbon source, accumulate a large amount of storage lipids.

In the invention, in order to produce a large amount of lipids, in a first step, the bacteria are cultured in a medium enriched with nitrogen and/or with phosphate for a predetermined period of time (preculture). In the invention, the phosphate or nitrogen sources are organic or mineral sources. This first culture is carried out for a short period of time and is considered to be a “preculture”. The bacteria are then transferred into a second medium which still comprises glycerol as the main carbon source, but which is nitrogen- and/or phosphate-poor. This second culture is the main culture.

The organic or mineral nitrogen and phosphate will subsequently be uniformly referred to as nutrients. Likewise, in the invention, the terms inorganic or mineral will be uniformly used to denote the nutrients which are not organic.

It is advantageous to carry out the preculture in a medium enriched with nitrogen and/or with phosphate, and to carry out the culture in a medium which is poor with respect to the same nutrients.

Thus:
if the preculture is enriched with nitrogen, the culture will be carried out in a nitrogen-poor medium,
if the preculture is enriched with mineral or organic phosphate, the culture will be carried out in a mineral or organic phosphate-poor medium, and
if the preculture is enriched with mineral or organic phosphate and with nitrogen, the culture will be carried out in a medium which is mineral or organic phosphate-poor and nitrogen-poor.

Culturing in a nutrient-rich medium and then in a nutrient-poor medium is known from the prior art as “hypercompensation”.

In the context of phosphate hypercompensation, from 2.5 to 50 mM of inorganic phosphate, preferentially from 3 to 40 mM, more particularly from 4 to 30 mM, even more particularly from 5 to 20 mM, in particular from 5 to 10 mM of inorganic phosphate is advantageously added to the preculture medium.

The organic phosphate added to such a preculture medium is done so by means of the following compounds: phosphate borne by nucleotides derived from nucleic acids such as ribonucleic acid (RNA), or deoxyribonucleic acid (DNA), or adenosine triphosphate (ATP), guanosine triphosphate (GTP), yeast extract, etc., this list not being limiting, and a person skilled in the art will be capable of determining the appropriate organic phosphate source.

The mineral phosphate used in the preculture medium is done so by means of the following soluble compounds: K₂HPO₄/KH₂PO₄ or Na₂HPO₄/NaH₂PO₄. In the preculture medium, the concentration of inorganic phosphate ranges from 4 mM to 20 mM, in particular the concentration is approximately 5 mM.

Advantageously, the concentration of glycerol in the preculture and the culture does not exceed 2M, advantageously 1.5M, in particular 1 M.

In the examples hereinafter, it is shown that the production of lipids by the actinomycetes correlates with the concentration of glycerol present in the culture and preculture medium. It will be considered that there is a glycerol-dose-dependent production of lipids up to a plateau reached at around 2M glycerol.

The use of glycerol as the main carbon source of the invention is more advantageous than the conventional use of glucose. This is because, as is shown in the examples hereinafter, the production of lipids by actinomycetes is dependent on the dose, or concentration, of glycerol, whereas strong concentrations of glucose have the effect of inhibiting growth and the production or accumulation of lipids.

In the invention, the term “main carbon source” is intended to mean an amount of carbon-based compound(s) which can be metabolized by the bacteria and which makes it possible to produce energy, in particular in the form of adenosine triphosphate (ATP), and which allows the biosynthesis of the organic molecules required for the growth and multiplication of said bacteria. The main carbon source represents more than 90% of the carbon supply in the culture or preculture medium.

In the invention, the term “C₃-C₅ sugars” is intended to mean sugars comprising 3 or 4 or 5 carbons. These are both aldoses (polyalcohols comprising an aldehyde function) or ketoses (polyalcohols comprising a ketone function), said C₃, C₄ and C₅ sugars being in their linear or cyclic form, as appropriate, and optionally modified.

In the invention, the lipids thus produced by the actinomycetes using glycerol or a C₃-C₅ sugar are produced in the form of a composition comprising a mixture of fatty acids, said mixture comprising essentially triacyl glycerols or TAGs.

The expression “composition comprising essentially triacyl glycerols” is intended to mean an oleaginous composition comprising more than 75% of triacyl glycerols, especially more than 80%, in particular more than 85%, more particularly more than 90%, relative to the total amount of lipid contained in said composition.

The composition also comprises minor amounts of free fatty acids, monoacyl glycerols and diacyl glycerols.

The lipid-producing actinomycetes according to the invention are essentially bacteria belonging to the following genera: Streptomyces, Rhodococcus, Amycolatopsis, Actinomyces, Arthrobacter, Corynebacterium, Frankia, Micrococcus, Micromonospora, Mycobacterium, Nocardia, Propionibacterium, etc.

In one advantageous embodiment, the invention also relates to the use as defined above, in which the culturing in the first medium is carried out for a period of time of from 15 to 120 hours.

The preculture is advantageously carried out for a period of time of approximately 15 to 120 hours, in particular from 20 to 90 hours, more particularly from 24 to 60 hours, in particular from 36 to 48 hours. The preculture can therefore be carried out for 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119 or 120 hours.

The culture, in the nutrient-poor medium, as was defined above, is carried out for a period of time of approximately 15 to 120 hours, advantageously from 20 to 80 hours, in particular 72 hours.

When the culture medium is inorganic phosphate-poor, the concentration of said phosphate ranges from 0.5 mM to 2 mM, and in particular is 1 mM.

In one advantageous embodiment, the invention relates to the use previously defined, where said C₃-C₅ sugar is a triose, a tetrose or a pentose.

In another advantageous embodiment, the invention also relates to the use as defined previously, in which the C₃-C₅ sugars are: glyceraldehyde, erythrose, threose, ribose, arabinose, xylose, lyxose, dihydroxyacetone, erythrulose, ribulose and xylulose.

In yet another advantageous embodiment, the invention relates to the abovementioned use, where said bacteria are actinomycetes of the Streptomyces genus.

In yet another advantageous embodiment, the invention relates to the abovementioned use, in which the glycerol is chosen from refined glycerol and unrefined glycerol.

Although unrefined glycerol is advantageous, because of the production costs, it is also advantageous to use essentially pure or refined glycerol, i.e. glycerol which is approximately at least 90%, especially 95%, in particular 99%, especially 100% enriched.

Various methods are known for producing glycerol.

The historical synthesis of glycerin or glycerol is due to Wurtz, starting from allyl tribromide. However, this synthesis is not total since allyl tribromide is itself prepared from glycerin. Total synthesis is due to Charles Friedel and Silva starting from propylene.

Various routes of synthesis starting from propylene exist. The epichlorohydrin process is the most important; it comprises the chlorination of propylene to give allyl chloride which is oxidized with hypochlorite to dichlorohydrin, which reacts with a strong base to give epichlorohydrin. The epichlorohydrin is then hydrolyzed to give glycerol.

Glycerol can also be produced by fermentation methods, involving for example yeasts, starting from monosaccharides, by:
(1) formation of a complex between acetaldehyde ions and bisulfite ions thus delaying the production of ethanol and restoring the oxidation-reduction balance in the synthesis of glycerol, or
(2) cultures of microorganisms growing at pH values close to 7 or above, or
(3) using osmotolerant microorganisms.

However, today, glycerol is produced industrially according to two main processes: saponification of fatty substances or transesterification of vegetable oils.

The saponification reaction is a reaction which makes it possible to produce soap and glycerol from fatty substances and sodium hydroxide. The glycerol resulting from saponification is very pure (>99%) and, as a result, is mainly used for pharmaceutical or cosmetic applications.

Glycerol, or glycerin, is also produced by transesterification of vegetable oils, of which it is a co-product, during the production of vegetable oil methyl esters (VOMEs) which are used as fuels under the name biodiesel.

Transesterification is a reaction comprising three reversible steps in series in which triglycerides are converted to diglycerides, then the latter are converted to monoglycerides and then, finally, the monoglycerides are converted to esters (biodiesel) and glycerol (co-product).

During the transesterification reaction, the oils or fats react with a short-chain alcohol (generally methanol) in the presence of a catalyst.

This reaction can be carried out by homogeneous catalysis, with catalysts that are soluble in the reaction medium, or by heterogeneous catalysis, with catalysts that are totally insoluble in the reagents.

At the current time, homogeneous catalysis is the technique most generally used in methods for producing biodiesel. The transesterification can be carried out by basic or acid catalysis. A greater reactivity is generally obtained in a basic medium.

Three major categories of catalysts exist:
basic catalysts:
   hydroxides, alkoxides or soaps of alkali or alkaline-earth metals (Li, Na, K, Ca, Ba, Cs, etc.),
   amines of the guanidine family, for example;
acid catalysts:
   mineral acids: HCl, H₂SO₄,
   sulfonic acids,
   ion exchange resins (strong acid),
   zeolites;
other catalysts:
   titanium alkoxides: Ti(OBu)₄, Ti(OPr)₄, etc.,
   oxides of various metals such as Sn, Mg, Zn, Ti, Pb, etc.

Acid catalysts are rarely used owing to their lower reactivity and the high risks of corrosion of the industrial equipment. Metal alkoxides or oxides are especially used for the synthesis of esters of heavy alcohols from various fractions of fatty acid methyl esters.

Sodium hydroxide in methanolic solution or sodium methoxide are the catalysts retained for biodiesel production.

The chemical composition of unrefined glycerol varies according to the production method. Thus, components such as methanol, various salts or potassium chloride may be found therein with variable contents in the final product.

Generally, whatever the production method used, glycerol represents from 63% to 99% of the total product. The methanol content ranges from 0 to approximately 25%. The phosphorous and potassium contents are between 1% and 2% of the solids content. Sodium is present in a proportion of 1% of the solids content.

Heterogeneous catalysis has significant advantages in terms of environmental friendliness limiting waste, and a neutral glycerol free of salts or water results from such a method, for example the one described in patent FR-B-2 752 242. The absence of salts in the reaction products means that, unlike homogeneous catalysis, purification treatments are not necessary.

Other techniques make it possible to carry out this reaction using innovative technologies such as microwave heating, using enzymatic catalysis, or else using supercritical methanol.

The crude glycerol produced can be purified or refined by treatment with active carbon to remove the organic impurities, by alkaline treatment to remove the glycerol esters which have not reacted, and by ion exchange to remove the salts. The glycerol obtained by means of a series of distillations has a high purity (>99.5%).

In yet another advantageous embodiment, the invention relates to the abovementioned use, wherein said bacteria are lipogenic bacteria.

The term “lipogenic bacteria” is intended to mean in the invention bacteria which are capable of naturally producing at least 20% of their biomass in lipids.

Such bacterial strains are advantageous since they make it possible, when they are cultured according to the method of the invention, to produce large amounts of lipids, representing more than 50% of their biomass.

Advantageous lipogenic strains of the invention are the following strains: Streptomyces antibioticus 3137, Streptomyces peuceius, Streptomyces rimosus 2535, Streptomyces coelicolor ATCC 19832, Streptomyces coelicolor ATCC21666, Streptomyces coelicolor Variant M145, Streptomyces coelicolor Variant M145M1155, Streptomyces coelicolor Variant M145M1146, Streptomyces coelicolor Variant M145M1141, Streptomyces coelicolor Variant M145M1148, Streptomyces coelicolor Variant M145M1142 and Streptomyces coelicolor Variant M145M1144, Streptomyces linolmensis NRRL2936, Streptomyces exfoliatus, Streptomyces cealestius ATCC 15084, Streptomyces lividans 1326, Streptomyces actuosus, Streptomyces ambofaciens OS MAROC J9 and J5, Streptomyces reticuli DSM 40776, Streptomyces parvulus 2283FB, Streptomyces venezuela 5110, Streptomyces noursei and Streptomyces cinabarinnus.

Another advantageous embodiment of the invention relates to the abovementioned use, wherein said lipogenic bacteria are genetically modified by substitution, deletion or insertion of at least one nucleic acid of their genome, such that they accumulate more lipids than the original strain.

For example, the inventors have shown that deleting the two components AbsA1/AbsA2 of the pathway for the biosynthesis of the peptide-type “calcium dependent antibiotic” results in an increase in the accumulation of triacyl glycerols (TAGs) in Streptomyces, under the conditions as described above (i.e. according to the method of the invention).

The inventors have also noted that the Streptomyces coelicolor M145 strain in which the four major pathways for antibiotic biosynthesis have been deleted (CPK, CDA, RED and ACT) and wherein two point mutations A262G and C271T have been introduced into the rpsL gene (SCO4659, 30S ribosomal protein S12); variant strain M145: M1155, exhibits increased lipid production compared with M145, this being especially in the presence of glucose. In the presence of glycerol, the lipid accumulation is strong even in the original M145 strain and the various genetic modifications introduced have a much weaker impact than in glucose.

The S. coelicolor M145 strain accumulates approximately 30%/6% of its biomass in TAGs when it is cultured in R2YE medium containing 0.2M glycerol/0.1M glucose for 96 h, respectively.

The variant strain of M145, M1155, described in Gomez-Escribano and Bibb, Microbial Biotechnology (2011) 4(2), 207-215 accumulates 40% of its biomass in TAGs when it is cultured in R2YE medium containing 0.2M glycerol, under hypercompensation conditions.

In the light of the knowledge regarding bacterial genomes, and of the knowledge of those skilled in the art regarding antibiotic biosynthesis pathways, those skilled in the art can easily determine the genes or gene sequences which must be targeted.

Moreover, the rpoB and/or rpsL genes can be mutated (by point mutation). These mutations have a positive effect both on the accumulation of storage lipids and on the production of antibiotics (in particular of polyketide type), suggesting that these mutations would have a positive effect on the generation of acetylCoA.

Furthermore, the genes or gene combinations which participate in the production of storage lipids can also be mutated:
the genes involved in increasing the influx (transport) of glycerol, its conversion to glycerol 3P (TAG precursor) and its catabolism to acetylCoA (enzymatic and/or signaling/regulation functions),
the genes involved in the biosynthesis of TAGs, the biosynthesis of fatty acids and their loading onto the glycerol backbone (enzymatic and/or signaling/regulation functions),
the genes involved in TAG degradation.

In the context of the abovementioned use, it is also advantageous to use bacterial strains exhibiting gene modifications which increase the availability of precursors for lipid synthesis. Thus, for example, it is advantageous to have bacterial strains which accumulate intermediate metabolites such as glycerol 3-phosphate (G3P) or else acetyl and malonyl coenzyme A (acetylCoA, malonylCoA).

In the context of the abovementioned use, it is also advantageous to use bacterial strains exhibiting gene modifications which increase the availability of precursors of lipid synthesis. Thus, for example, it is advantageous to have bacterial strains which accumulate intermediate metabolites such as glycerol 3-phosphate (G3P) or else acetyl coenzyme A (acetylCoA).

In addition, in the context of the abovementioned use, it is advantageous to use bacterial strains exhibiting gene modifications which increase the activity of the enzymatic systems involved in the generation of precursors (acetyl and malonylCoA) and the biosynthesis of TAGs, among which mention may be made of the increase in the availability of co-factors essential to enzymatic activity (biotin, etc.) or in the intensity of post-translational modifications (phosphorylation, acetylation, glycosylation, etc.) which have a positive effect on the activity of the enzymes which play a role in precursor generation and TAG biosynthesis.

Glycerol can be metabolized via two potentially concurrent pathways which both result in the synthesis of acetylCoA:
the glycerol kinase, Gly3P DH (operon SCO1659 to SCO1661 under the control of the GylR repressor, SCO1658) and SCO4774 (putative glycerol 3P dehydrogenase) pathway. This pathway results in the production of Gly3P required for TAG biosynthesis.

In order to increase the G3P pool, it is in particular possible to jointly overexpress the SCO1659 (glycerol transporter) and SCO1660 (glycerol kinase) genes or even to inactivate SCO4774 which is supposed to convert Gly3P to DHAP which enters glycolysis;
the glycerol dehydrogenase (SCO2598/SCO6754), DHA kinase (SCO0580, operon SCO0581 to SCO0576, with regulator divergent, SCO0582) and/or operon SCO7073 to SCO7071 pathway.

It is also possible to envision increasing the catabolism of glycerol via the glyDH/DHA kinase pathway in order to increase the acetylCoA pool.

In any event, particular attention will be given to the redox equilibrium of the cell (the NADH generated will have to be reoxidized).

In order to increase the acetylCoA pool, it is also possible to increase the expression of the genes encoding the enzymes of glycolysis and/or the activity of the corresponding enzymes by adjusting any post-translational regulations and/or the abundance of cofactors required for the activity of these enzymes. Particular interest will be given to the enzymes of the lower part of glycolysis and in particular to the pyruvate dehydrogenase complex.

In order to increase the production of lipids by bacteria, it is also advantageous to carry out genetic modifications in said bacteria in order to reduce fatty acid degradation.

By way of illustration, it may be advantageous to reduce the expression of the genes encoding the enzymes of the glyoxylate pathway. In particular, the SCO0982 gene and the neighboring genes can be deleted or mutated such that their products are no longer synthesized or are no longer functional.

The abovementioned examples of gene modifications are given only by way of indication and should not be considered to limit the scope of the invention. Those skilled in the art, with their general knowledge of the field in question, are capable of determining the most appropriate modifications.

The description also relates to a set of culture media comprising glycerol, or a C₃-C₅ sugar, as the main carbon source, said set of culture media comprising:
a first culture medium enriched with mineral phosphate and/or with nitrogen, and
a second culture medium which is mineral phosphate-poor and/or nitrogen-poor.

Furthermore, the description relates to a set of culture media comprising glycerol, or a C₃-C₅ sugar, as the main carbon source, said set of culture media comprising:
a first culture medium enriched with mineral phosphate and/or with nitrogen, and
a second culture medium which is mineral phosphate-poor and/or nitrogen-poor.

Advantageously, the invention also relates to a set of culture media comprising glycerol, or a C₃-C₅ sugar, as the main carbon source, said set of culture media comprising:
a first culture medium enriched with mineral phosphate, and
a second culture medium which is mineral phosphate-poor.

Advantageously, the description also relates to a set of culture media comprising glycerol, or a C₃-C₅ sugar, as the main carbon source, said set of culture media comprising:
a first culture medium enriched with nitrogen, and
a second culture medium which is nitrogen-poor.

In the invention, the culture media are media commonly used by those skilled in the art to enable the growth of actinomycete bacteria.

By way of example, in the context of the culture of streptomycetes, the HT, YEME, MP5, SL11, R2 or R2YE and modified YEME media can be used. The compositions of various advantageous media are described in the example section hereinafter.

Advantageously, in the invention, the first culture medium and the second culture medium are of the same nature. Thus, if the first medium is R2YE medium, the second medium will advantageously be R2YE medium, optionally slightly modified, in particular no longer comprising sucrose.

In certain aspects of the invention, it may be opportune to choose a first medium of different nature to the second medium. Those skilled in the art will be able to choose the most appropriate media according to the bacterial strains used.

The description relates, moreover, to a set comprising:
a first culture medium enriched with phosphate and/or with nitrogen, and
a second culture medium which is phosphate-poor and/or nitrogen-poor, said first and second culture media comprising, as the main carbon source, glycerol, or a C₃-C₅ sugar, in particular a sugar chosen from glyceraldehyde, erythrose, threose, ribose, arabinose, xylose, lyxose, dihydroxyacetone, erythrulose, ribulose and xylulose, and
at least one strain of actinomycete bacteria.

Advantageously, the invention relates, moreover, to a set comprising:
a first culture medium enriched with phosphate, and
a second culture medium which is phosphate-poor, said first and second culture media comprising, as the main carbon source, glycerol, or a C₃-C₅ sugar, in particular a sugar chosen from glyceraldehyde, erythrose, threose, ribose, arabinose, xylose, lyxose, dihydroxyacetone, erythrulose, ribulose and xylulose, and
at least one strain of actinomycete bacteria.

Advantageously, the description relates, moreover, to a set comprising:
a first culture medium enriched with nitrogen, and
a second culture medium which is nitrogen-poor,
   said first and second culture media comprising, as the main carbon source, glycerol, or a C₃-C₅ sugar, in particular a sugar chosen from glyceraldehyde, erythrose, threose, ribose, arabinose, xylose, lyxose, dihydroxyacetone, erythrulose, ribulose and xylulose, and
at least one strain of actinomycete bacteria.

As previously mentioned, the actinomycete bacteria are in particular lipogenic strains, in particular streptomyces, in particular chosen from Streptomyces antibioticus 3137, Streptomyces peuceius, Streptomyces rimosus 2535, Streptomyces coelicolor ATCC 19832, Streptomyces coelicolor ATCC21666, Streptomyces coelicolor Variant M145, Streptomyces coelicolor Variant M145M1155, Streptomyces coelicolor Variant M145M1146, Streptomyces coelicolor Variant M145M1141, Streptomyces coelicolor Variant M145M1148, Streptomyces coelicolor Variant M145M1142 and Streptomyces coelicolor Variant M145M1144, Streptomyces linolmensis NRRL2936, Streptomyces exfoliatus, Streptomyces cealestius ATCC 15084, Streptomyces lividans 1326, Streptomyces actuosus, Streptomyces ambofaciens OS MAROC J9 and J5, Streptomyces reticuli DSM 40776, Streptomyces parvulus 2283FB, Streptomyces venezuela 5110, Streptomyces noursei and Streptomyces cinabarinnus.

In addition, the description relates to a method for the production of lipids by actinomycete bacteria, said method comprising
a step of culturing said bacteria in a first medium enriched with phosphate and/or with nitrogen, and
a step of culturing said bacteria in a second medium which is phosphate-poor and/or nitrogen-poor,
   said first and second culture media comprising, as main/principal carbon source, glycerol or a C₃-C₅ sugar.

Advantageously, the invention relates to a method for the production of lipids by actinomycete bacteria, said method comprising
a step of culturing said bacteria in a first medium enriched with phosphate, and
a step of culturing said bacteria in a second medium which is phosphate-poor,
   said first and second culture media comprising, as main/principal carbon source, glycerol or a C₃-C₅ sugar.

Advantageously, the description relates to a method for the production of lipids by actinomycete bacteria, said method comprising
a step of culturing said bacteria in a first medium enriched with nitrogen, and
a step of culturing said bacteria in a second medium which is nitrogen-poor,
   said first and second culture media comprising, as main/principal carbon source, glycerol or a C₃-C₅ sugar.

Advantageously, the invention relates to a method as defined previously, in which said bacteria are actinomycetes of the streptomycete family.

In one advantageous embodiment, the invention relates to a method as defined previously, wherein said bacteria are lipogenic bacteria, in particular bacteria genetically modified by substitution, deletion or insertion of at least one nucleic acid of their genome, such that they accumulate more lipids than the original strain.

The invention relates, moreover, to a lipid composition comprising lipids which can be obtained by means of the method as defined above.

An advantageous composition of the invention, which can be obtained by means of the abovementioned method, comprises, or essentially consists of, or consists of, the following fatty acids:
from 6.7% to 7.7% of iso C14:0,
from 3.1% to 4% of C14:1,
from 9.8% to 11% of antiiso C15:0,
from 0.1% to 0.7% of C15:0,
from 61.5% to 62.5% of iso C16:0,
from 0.5% to 1.5% of iso C16:1,
from 3.1% to 4% of C16:0,
from 0.1% to 7.7% of C16:1,
from 0.5% to 1.5% of C17:0,
from 0.5% to 1.5% of C17:1,
from 1.1% to 2.1% of iso C17:0, and
from 6.7% to 7.7% of antiiso C17:0,
   the percentages being expressed by weight of the total fatty acids of the composition.

Moreover, in the composition according to the invention, the fatty acids are mainly in TAG form, i.e. at least 50% of the fatty acids are in TAG form.

In the invention, the fatty acids are the following:
12-methyltridecanoic acid (iso C14:0/iso-tetradecanoic acid),
tetradecenoic acid (C14:1),
12-methyltetradecanoic acid (antiiso C15:0/antiiso-pentadecanoic acid),
pentadecanoic acid (C15:0),
14-methylpentadecanoic acid (iso C16:0/iso-hexadecanoic acid),
14-methylpentadecenoic acid (iso C16:1/iso-hexadecenoic acid),
hexadecanoic acid (C16:0),
hexadecenoic acid (C16:1),
15-methylhexadecanoic acid (iso C17:0/iso-heptadecanoic acid),
heptadecanoic acid (C17:0),
heptadecenoic acid (C17:1), and
14-methylhexadecanoic acid (antiiso C17:0/antiiso-heptadecanoic acid).

The invention also relates to the use of the abovementioned method, for the production of lubricants, surfactants, coatings (paints, inks, etc.), solvents and food ingredients or as synthesis intermediates for oleochemistry.

It is possible, for example, to obtain biodiesel by means of known methods of trans-esterifications, for example a method of trans-esterification of the triglycerides present in the composition according to the invention or the microorganism extract according to the invention with methanol in the presence of a catalyst, in order to obtain the methyl esters of fatty acids and of glycerol. The biodiesel obtained can be used as a mixture with diesel of fossil origin or pure, as a fuel.

It is also possible to produce biokerosene by means of a treatment with hydrogen. This method consists in a first step aimed at removing the oxygen present in the feedstocks and in converting them into a fraction composed of paraffinic hydrocarbons, and a route termed decarboxylation route, which results in hydrocarbons containing one carbon atom less than the initial fatty chain, and which is accompanied by the formation of CO and CO₂. In any event, the unsaturations of the fatty chains are totally hydrogenated, and the glyceryl group is converted to propane. Moreover, side reactions, such as shift and methanation reactions, can result in the formation of CO and of methane. On the catalysts of sulfide type conventionally used in hydroprocessing, the two routes of oxygen elimination (hydrogenation and decarboxylation) coexist.

The hydrogen consumptions corresponding to this hydrodeoxygenation step depend on the nature of the starting animal fats or oil, in particular on the average number of unsaturations per chain and on the length of the hydrocarbon-based fatty chains.

Next, the isomerizing hydrocracking step makes it possible to crack the paraffinic diesel chains to give branched kerosene and naphtha. The kerosene yield regarding diesel is approximately 60% (40% of naphtha). In the prior art, the overall kerosene yield regarding crude oil is about 55%.

It is also possible to obtain lubricants, solvents and surfactants or food products from the abovementioned method, by transesterification or hydrolysis which is chemical or enzymatic and/or combined with other chemical processes.

All of these techniques are well known to those skilled in the art.

The description also relates to a method for the production of biofuel, lubricants, surfactants, coatings (paints, inks, etc.), solvents and synthesis intermediates from lipids derived from actinomycete bacteria, said method comprising
a step of culturing the bacteria in a first medium enriched with phosphate and/or with nitrogen, and
a step of culturing said bacteria in a second medium which is phosphate-poor and/or nitrogen-poor,
   said first and second culture media comprising, as the main carbon source, glycerol or a C₃-C₅ sugar.

The invention also relates to a method for the production of biofuel, lubricants, surfactants, coatings (paints, inks, etc.), solvents and synthesis intermediates from lipids derived from actinomycete bacteria, said method comprising:
a step of culturing the bacteria in a first medium enriched with phosphate, and
a step of culturing said bacteria in a second medium which is phosphate-poor, said first and second culture media comprising, as the main carbon source, glycerol or a C₃-C₅ sugar.

The lipid composition obtained according to the method of the invention can be used to produce a biofuel using the method described in application WO 2005/093015.

By way of indication, the method for the production of a lipid composition that can be used as a biofuel or as a fuel constituent, from the composition obtained by means of the method of the invention, comprises
at least one transesterification step in which the composition obtained by means of the method according to the invention is reacted by heterogeneous catalysis with at least one primary monoalcohol chosen from methanol and ethanol, so as to give, on the one hand, at least one methyl and/or ethyl ester of the starting triglyceride fatty acid(s) and, on the other hand, glycerol, these products being free of by-products; and
an etherification step in which the glycerol is reacted with at least one olefinic hydrocarbon having from 4 to 12 carbon atoms; and/or
an acetalization step in which the glycerol is reacted with at least one compound chosen from aldehydes, ketones and acetals derived from aldehydes or from ketones.

Two types of catalysis can be envisioned for carrying out the transesterification of a vegetable oil to methyl (or ethyl) esters using heterogeneous catalysts: a batch-reactor catalysis or a continuous catalysis using the fixed-bed principle. The process is generally carried out continuously in a fixed bed.

The abovementioned method is given by way of example and should not be considered to be limiting. Those skilled in the art will be capable of using any other similar method with the aid of their general knowledge in the field.

The invention will be understood more clearly with the aid of the examples and the seven figures which follow.

### BRIEF DESCRIPTION OF THE DRAWINGS

FIG. 1 is a graph representing the percentage proportion of TAGs accumulated by strain. The strains tested are the following: A: S. antibioticus 3137 J II, B: S. peucetius, C: S. rimosus 2535 JII, D: S. coelicolor Variant M145 M 1155, E: S. lincolnensis NRRL 2936, F: S. exfoliatus, G: S. avermitilis NRRL 8165, H: S. coelicolor Variant M145 M1146, I: S. coelicolor Variant M145 M1141, J: S. coelicolor Variant M145 M1148, K: S. cealestis ATCC 15084, L: S. lividans 1326, M: S. coelicolor Variant M145 M1142, N: S. actuosus, O: S. ambofaciens. OS. MAROC J 9, P: S. reticuli DSIVI 40776, Q: S. ambofaciens.05.MAROC J 5, R: S. parvulus 2283 FB, S: S. coelicolor Variant M145 M1144, T: S. coelicolor M145, U: S. coelicolor ATCC 19832, V: S. venezuela 5110J11, W: S. noursei, X: S. cinabarinnus ATCC 23617, Y: S. coelicolor ATCC 21666 and Z: S. venezuelae ATCC 15439.

FIGS. 2A-C represent the accumulation of TAGs by Streptomyces lividans TK24. FIG. 2A shows an electron microscopy photo of a mycelium of Streptomyces lividans cultured for 48 hours, from spores, on an R2YE medium in the presence of glycerol and of inorganic phosphate (5 mM).

FIG. 2B shows an electron microscopy photo of a mycelium of Streptomyces lividans resulting from the step described in FIG. 2A, and cultured for 48 hours on a new R2YE medium which is in the presence of glycerol and is inorganic phosphate-poor (5 mM).

FIG. 2C shows an electron microscopy photo of a mycelium of Streptomyces lividans resulting from the step described in FIG. 2A, and cultured for 48 hours on a new R2YE medium which is in the presence of glycerol and is inorganic phosphate-poor (1 mM).

The presence of accumulated lipids is identified by the presence of white lipid vesicles.

FIG. 3 represents a graph showing the ratio of the TAG carbonyl band/amide band of the proteins, which corresponds to the percentage of TAGs relative to the dry weight, of various strains of Streptomyces (1: S. coelicolor M145, 2: S. coelicolor M1146, 3: S. coelicolor M1155, 4: S. antibioticus and 5: S. rimosus) cultured for 72 h on an R2YE medium in the presence of 0.1M of glucose (white bars) or in the presence of 0.2M of glycerol (black bars).

FIG. 4 represents a graph showing the ratio of the TAG carbonyl band/amide band I of the proteins, which corresponds to the percentage of TAGs relative to the dry weight, of a strain of Streptomyces (S. lividans TK24) cultured in the presence of various carbon sources (of C₃ or C₅) (1: 0.2M glycerol, 2: 0.17M xylose and 3: 0.17M arabinose) for 72 h on an R2YE medium. The same amounts of carbon equivalent were used.

FIGS. 5A-C represent the fatty acid composition of the lipids accumulated under the conditions of the invention, during a culture in the presence of glucose, glycerol, or refined glycerol.

FIG. 5A represents a graph showing the proportion, in μg per mg of dry biomass, of each of the fatty acids indicated, after culture for 96 h in the presence of 0M of glucose (white bars), 0.1M of glucose (black bars) and 0.2M of glycerol (hatched bars) of a strain of Streptomyces.

FIG. 5B represents a graph showing the proportion, in μg per mg of dry biomass, of each of the fatty acids indicated, after culture for 96 h in the presence of 0 M of refined glycerol (white bars), 0.1M of glycerol (black bars) and 0.2M of glycerol (hatched bars) of a strain of Streptomyces.

FIG. 5C represents a graph showing the proportion, in μg per mg of dry biomass, of each of the fatty acids indicated, after culture for 96 h in the presence of 0 M of refined glycerol (white bars), 0.1M of refined glycerol (black bars) and 0.2M of refined glycerol (hatched bars) of a strain of Streptomyces.

FIGS. 6A-B illustrate the advantageous effect of mutations on the lipid production in R2YE glucose medium.

FIG. 6A shows diagrammatically the genes involved in lipid degradation via the glyoxylate pathway, said pathway being present essentially in oleaginous microorganisms.

FIG. 6B represents a graph showing TAG accumulation in strains of Streptomyces lividans which have a deletion of at least one gene of the glyoxylate pathway.

FIG. 7 represents a graph showing the ratio of the TAG carbonyl band/amide band I of the proteins, which corresponds to the percentage of TAGs relative to the dry weight, of a strain of Streptomyces (S. coelicolor M1155) cultured on a medium comprising, as carbon source, glucose (white columns) or glycerol (black columns), the carbon sources being at concentrations of 1: 18 g/l, 2: 45 g/l, 3: 68 g/l and 4: 90 g/l, for 96 h on a sucrose-free R2YE medium.

### EXAMPLES

### Example 1

### Culture Media

The following culture media can be used in the context of the invention. All of the culture media described hereinafter are supplemented with C₃-C₅ sugars, with glucose or with glycerol:glucose (10 g/l or 18 g/l) or glycerol (0.2 mol/l and 18.4 g/l).

### HT Culture Medium:

The HT medium comprises, for one liter: 1 g of yeast extract (yeast extract-Difco), 1 g of beef extract (Difco), 10 g of white dextrin (Prolabo), 2 g of NZ amine type A, 20 mg of CoCl₂.7H₂O.

### Modified R2YE Culture Medium:

K₂SO₄ (1.4×10⁻³ mol/l and 0.25 g/l) MM=174 g/mol

MgCl₂.6H₂O (5×10⁻² mol/l and 10.12 g/l) MM=203 g/mol

Casein amino acids (0.1 g/l)

CaCl₂.2H₂O (2×10⁻² mol/l and 2.94 g/l) MM=147 g/mol

L-proline (2.6×10⁻² mol/l and 3 g/l) MM=115 g/mol

TES buffer (2.5×10⁻² mol/l and 5.73 g/l) MM=2304 g/mol

Trace elements (2 ml/l)

Yeast extract (5 g/l)

NaOH (5×10⁻³ mol/l and 0.2 g/l) MM=30 g/mol

R2YE trace element stock solution:

ZnCl₂.40 mg/l; FeCl₃.6H2O 200 mg/l; CuCl₂.2H₂O 10 mg/l; MnCl₂.4H₂O 10 mg/l; Na₂B₄O₇.10H₂O 10 mg/l; (NH₄)₆Mo₇O₂₄.4H₂O 10 mg/l

### Medium 194 Synthetic Medium:

TES 22.8 mM;

Citric acid.H₂O 2.0 mM;

NaCl 2.2 mM;

KH₂PO₄ 11.0 mM;

(NH₄)₂SO₄ 43.0 mM;

MgSO₄.7H₂O 1.7 mM;

CaCl₂.2H₂O 0.2 mM;

FeSO₄.7H₂O 137.6 μM;

CuSO₄.5H₂O 12.0 μM;

ZnSO₄.7H₂O 11.7 μM;

MnSO₄.H₂O 33.9 μM;

Na₂MoO₄.2H₂O 1.6 μM;

CoCl₂.6H₂O 3.2 μM;

KI 1.5 μM;

AlCl₃.6H₂O 1.3 μM;

H₃BO₃ 2.5 μM;

NiCl₂.6H2O 1.6 μM;

Thiamine-HCl 21.0 μM (7 mg/l);

Biotin 1.2 μM (0.30 mg/l);

Riboflavin 5.0 μM (2 mg/l);

Calcium pantothenic acid 8.4 μM (2 mg/l);

Folic acid 0.6 μM (0.25 mg/l);

p-Aminobenzoic acid 1.8 μM (0.25 mg/l);

Pyridoxine-HCl 9.7 μM (2 mg/l);

Nicotinamide 16.3 μM (2 mg/l);

Nicotinic acid 0.5 μM (0.0625 mg/l);

Antifoam 204 100.0 μl/l;

Pluronic F68 10% 0.5 ml/l.

### YEME Culture Medium:

3 g of yeast extract, 5 g of bactopeptone, 3 g of malt extract, and 5 mM of MgCl₂.

### DALP Culture Medium:

The DALP medium comprises, for one liter: 10 g of bactopeptone, 5 g of yeast extract and 10 g of white dextrin. The pH is adjusted to 7.0.

### MP5 Culture Medium:

The MP5 medium comprises, for one liter: 7 g of yeast extract, 5 g of NaCl, 36 ml of glycerol, and 20.9 g of MOPS. The pH is adjusted to 7.5.

### SL11 Culture Medium:

The SL11 medium comprises, for one liter: 25 g of white dextrin, 12.5 g of yeast extract, 1 g of MgSO₄, 1 g of KH₂PO₃, and 20.9 g of MOPS. The pH is adjusted to 7.1

### Modified YEME Culture Medium:

The modified YEME medium comprises, for one liter: 3 g of yeast extract, 5 g of bactopeptone and 3 g of malt extract.

### Example 2

### Culture Conditions

### Liquid Cultures (50 ml Flask, 2 l Fermenter)

Preculture inoculated with 10⁶ spores/ml and incubated for 48 h at 28° C., pH 7, with shaking at 180-200 rpm (revolutions per minute).

The culture is inoculated with a volume of preculture representing 2.5% of the final volume of the culture. The culture is incubated for 72 to 96 h at 28° C., pH 7, 180-200 rpm, for a flask (for a fermenter: 70% pO₂, shaking at 400-600 rpm).

The samples taken are then centrifuged, and the bacterial pellet is frozen at −80° C. and lyophilized in order to obtain the dry mass. It is used for the analysis of the TAG content by FTIRS (Fourier Transform Infra Red Spectroscopy).

### Solid Cultures (Petri Dishes)

A first Petri dish containing a sporulation medium (SFM: soy flour 20 g, mannitol 20 g, bacto agar 15 g, tap water qs 1 l) is inoculated using spores in order to obtain a bacterial layer. This Petri dish is incubated for between 5 and 10 days until sporulation of the strain occurs.

The culture dish is composed of 8 ml of R2YE medium, covered with a cellophane. This dish is inoculated using a wooden stick. 20 μl of water are deposited on the cellophane, the stick having previously touched the SFM dish containing the sporulated strain is saturated with “fresh spores” and then immersed in the 20 μl of water present on the R2YE dish and plated out over the entire surface of the cellophane. The culture dishes are then incubated for 72 to 96 h at 28° C. in the dark. At the end of the culture, the bacteria are recovered by scraping the cellophane, and then frozen at −80° C. and lyophilized for the analysis of the dry weight and of the TAG content by FTIRS.

### Example 3

### Lipogenic Strains

The inventors identified Streptomyces strains considered to be lipogenic, i.e. strains which produce more than 20% of their biomass in TAGs.

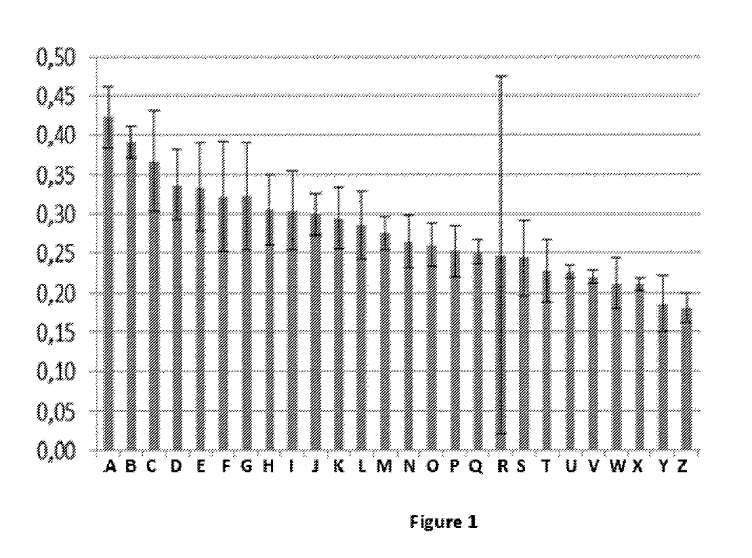

The results are presented in FIG. 1.

### Example 4

### Hypercompensation in Glycerol Medium

In order to determine the proportion of TAGs accumulated by the microorganisms, the inventors tested the Streptomyces lividans strain.

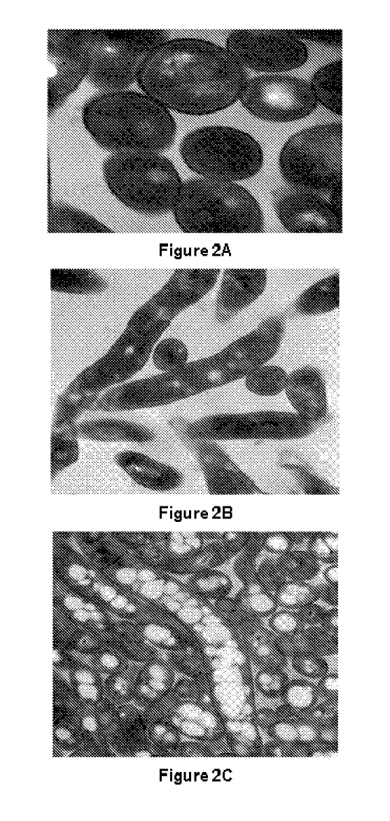

The bacteria were plated out at the surface of a cellophane deposited on solid R2YE medium containing glycerol (0.2M) and 5 mM of inorganic phosphate (Pi) for 48 h. Observation of the bacteria by electron microscopy shows that they contain very few lipid vesicles (FIG. 2A).

At the end of the 48 h, the bacteria were moved to the surface of a new solid R2YE medium, containing glycerol (0.2M) and either 5 mM of Pi (FIG. 2B, or 1 mM of Pi (FIG. 2C).

The electron microscopy observation shows that the bacteria that were subjected to a phosphate limitation (transfer from 5 mM to 1 mM Pi) very strongly accumulate TAGs (hypercompensation phenomenon), whereas those which were not subjected to such a limitation (transfer from 5 mM to 5 mM) do not show this phenomenon and accumulate a smaller amount of TAGs.

Similar results are obtained in the context of a nitrogen hypercompensation.

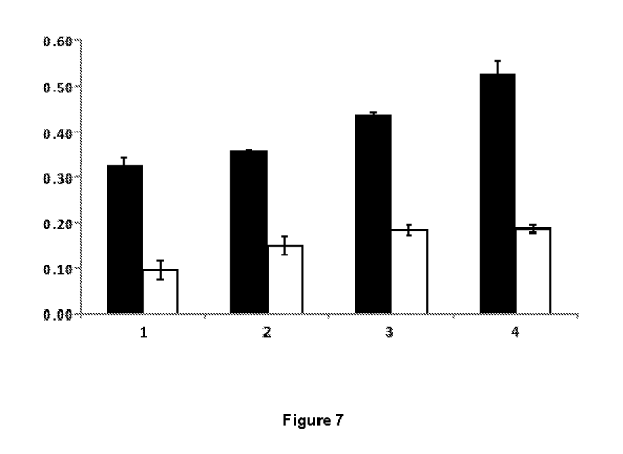

These results are confirmed with the Streptomyces coelicolor M1155 strain, as shown in FIG. 7. For this strain (M1155), it is noted that, in a dose-dependent manner, the bacteria cultured on R2YE/glycerol accumulate more TAGs than those cultured in R2YE/glucose.

### Example 5

### Hypercompensation in Glycerol Medium and in Glucose

In order to validate the previous results, the inventors compared the TAG accumulation under Pi hypercompensation conditions of various Streptomyces strains, in the presence of glucose (0.1M) or of glycerol (0.2M).

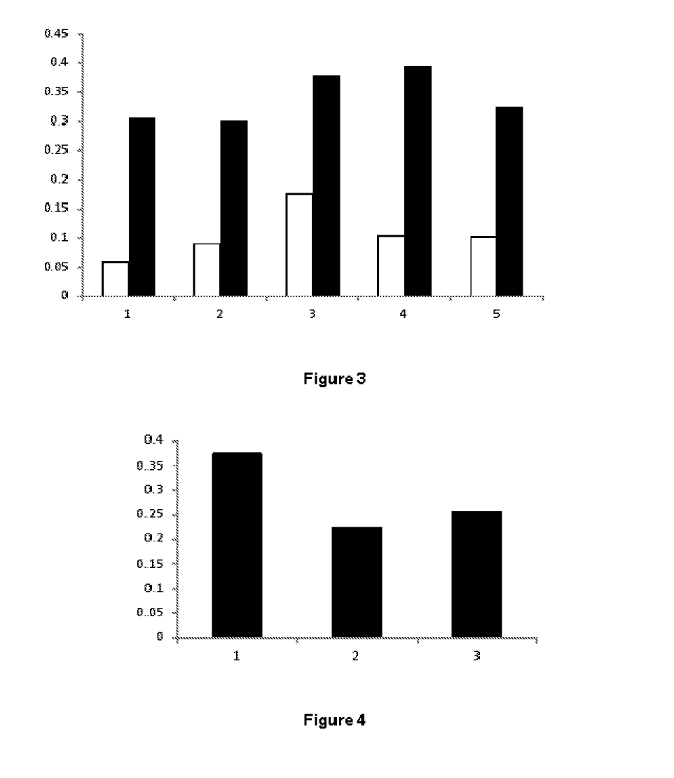

The results are presented in FIG. 3.

It is noted that, whatever the strain used, the bacteria cultured according to the method of the invention accumulate more TAGs than the same bacteria cultured in the presence of glucose.

### Example 6

### Hypercompensation in Medium Comprising C₅ Sugars

In order to validate the previous results, the inventors compared the TAG accumulation under Pi hypercompensation conditions of Streptomyces coelicolor in the presence of C₅ sugars (0.17 mM) or of glycerol (0.2M).

The cells are lyophilized and then crushed so as to have a powder which can be directly analyzed by FTIR spectrometry as described, for example, in Dean et al. 2010 Bioresource Technology, 101(12), 4499-4507.

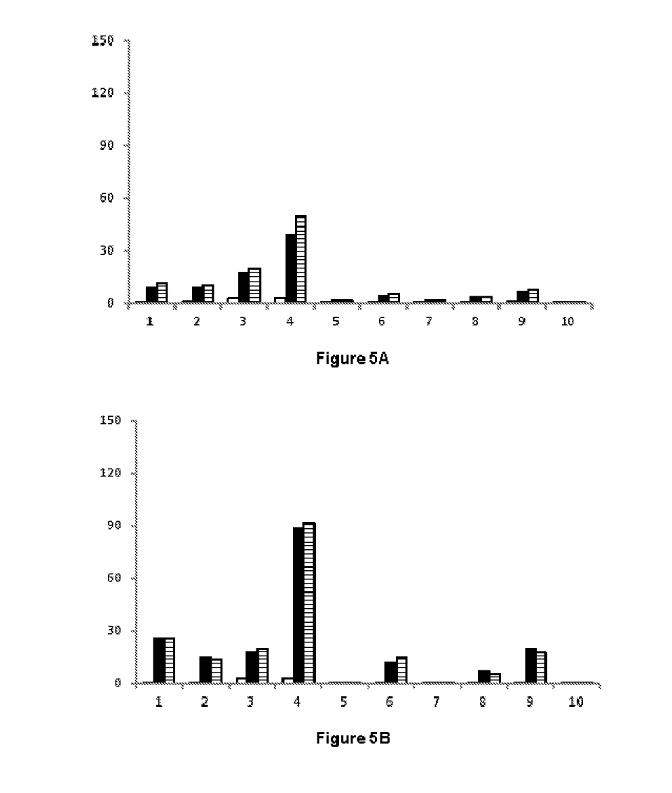

The results are represented in FIG. 4.

It is noted that the bacterium is capable of accumulating significant amounts of TAGs, including with C₅ sugars.

### Example 7

### Hypercompensation in Medium Comprising Refined or Unrefined Glycerol

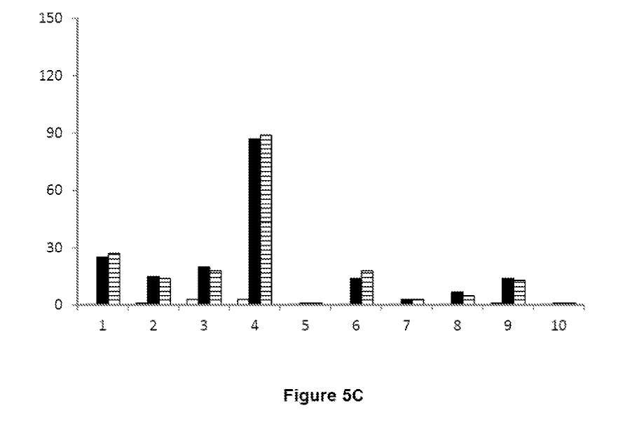

The inventors also identified the composition of fatty acids accumulated by the bacteria cultured under the conditions of the invention, in the presence of crude glycerol (FIG. 5B) or of refined glycerol (FIG. 5C), the glycerol being present at a concentration ranging from 0 to 0.2M. The same culture in the presence of glucose is used as a control (FIG. 5A).

These results show a greater accumulation when the glycerol source is not refined. Moreover, these results show that the TAGs which accumulate when the bacteria are cultured using the method according to the invention exhibit a high amount of iso C16:0 fatty acid, and to a lesser extent a not insignificant amount of antiiso C15:0. The quantification of the fatty acids accumulated in TAG form in the bacteria cultured in the presence of unrefined glycerol is the following: for 100 g of dry biomass:

iso C14:0: 3.5 g,

iso C14:1: 1.75 g,

antiiso C15:0: 5 g,

C15:0: 0.1 g,

iso C16:0: 30 g,

iso C16:1: 0.5 g,

C16:0: 1.75 g,

C16:1: 0.25 g,

iso C17:0: 0.8 g,

antiiso C17:0: 3.5 g,

C17:0: 0.5 g, and

C17:1: 0.5 g.

### Example 8

### Hypercompensation in Medium Comprising Glycerol and with a Fatty Acid Degradation Gene Deletion

In order to increase the TAG content of the bacteria, the inventors tested the impact of fatty acid degradation pathway modifications.

Bacteria (S. coelicolor M145) with deletions of various genes of the glyoxylate pathway were cultured under the conditions of the invention in the presence of glycerol.

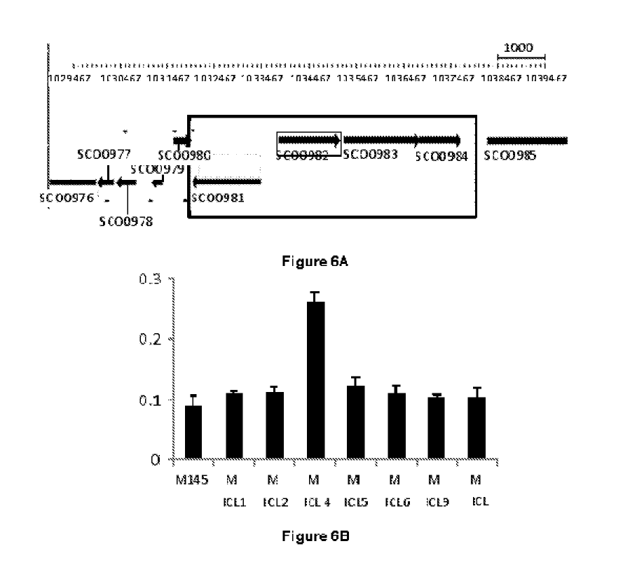

The results are presented in FIG. 6B.

It is noted that bacteria with a deletion of the SCO0981, SCO0982, SCO0983 and SCO0984 genes (ICL4 on FIG. 6B) accumulate up to more than 200% more TAGs than the bacteria which do not have such deletions.

These results show that the method according to the invention, combined with gene modifications of fatty acid metabolism pathways, makes it possible to considerably increase the fatty acid accumulation by the bacteria.

1. A method for the production of lipids by actinomycete bacteria, said method comprising
a step of culturing the bacteria in a first medium enriched with phosphate, and
a step of culturing said bacteria in a second medium which is phosphate-poor,
said first and second culture media comprising, as the main/principal carbon source, glycerol or a C₃-C₅ sugar.

2. The method as claimed in claim 1, wherein said carbon source is a triose, a tetrose or a pentose.

3. The method as claimed in claim 1, wherein said bacteria are actinomycetes of the Streptomyces genus.

4. The method as claimed in claim 1, wherein the glycerol is chosen from refined glycerol and unrefined glycerol.

5. The method as claimed in claim 1, wherein said bacteria are lipogenic bacteria.

6. The method as claimed in claim 5, wherein said lipogenic bacteria are genetically modified by substitution, deletion or insertion of at least one nucleic acid of their genome, such that they substantially do not produce antibiotic.

7. A set of culture media comprising glycerol, or a C₃-C₅ sugar, as the main carbon source, said set of culture media comprising:
a first culture medium enriched with phosphate, and
a second culture medium which is phosphate-poor.

8. A set comprising
a first culture medium enriched with phosphate, and
a second culture medium which is phosphate-poor,
said first and second culture media comprising, as the main carbon source, glycerol or a C₃-C₅ sugar, and
at least one strain of actinomycete bacteria.

9.-11. (canceled)

12. A lipid composition comprising lipids obtained by means of the method as defined in claim 1.

13. The method as claimed in claim 1, further comprising production of lubricants, surfactants, coatings, solvents, food ingredients or synthesis intermediates for oleochemistry from the lipids.

14. (canceled)
